(19) United States
(12) Patent Application Publication
(10) Pub. No.: US 2020/0246560 A1
(43) Pub. Date: Aug. 6, 2020

(54) IMPROVEMENTS IN METERED DOSE INHALER DEVICES

(71) Applicant: 3M INNOVATIVE PROPERTIES COMPANY

(72) Inventors: Peter D. HODSON, BREASTON (GB); Adam J. STUART, LOUGHBOROUGH (GB)

(21) Appl. No.: 15/776,162

(22) PCT Filed: Nov. 15, 2016
(86) PCT No.: PCT/US2016/062000

§ 371 Date: May 15, 2018

(30) Foreign Application Priority Data
Nov. 16, 2015 (GB) 1520177.5

(51) Int. Cl. A61M 15/00

(52) U.S. Cl. CPC A61M 15/009; A61M 2205/582; A61M 15/0021

(57) ABSTRACT

Described herein is an actuator housing (500) for use with a metered dose inhaler (MDI) device which comprises a housing portion (115) having a nozzle block in which an actuator seat and an actuator nozzle are formed, and a mouthpiece portion (525) joined to the housing portion (115). The mouthpiece portion (525) has an external surface having a first or guidance region (570) over which teeth of a patient are to be located and a second or sealing region (580) against which lips of the patient seal during dispensing of a medicament. In one embodiment, the first or guidance region (570) comprises a plurality of longitudinally extending flutes arranged around the external surface and located adjacent to a mouthpiece end face (560). An optional annular projection (590) may be provided to define more precisely the sealing region for the patient.

### FIELD OF THE INVENTION

The present invention relates to improvements in metered dose inhaler devices and is more particularly concerned with actuators for such devices.

### BACKGROUND OF THE INVENTION

All metered dose inhaler (MDI) actuators or devices for pulmonary delivery, whether they be standard “press-and-breathe” designs or designs employing automatic triggering, such as those found in the Autohaler™ or the Easibreathe™ devices, are designed to deliver an aerosol plume to the oral cavity at an angle which is essentially horizontal [Autohaler™ is a trademark of the 3M Corporation and Easibreathe™ is a trademark of Norton Healthcare Limited].

In real life dosing situations, delivery of the plume may vary from horizontal, due to, for example, face structure of a patient and/or inhaler technique. In addition, there is a possibility of the teeth and lips of the patient providing obstacles for the plume and further locations for deposition if the actuator is not positioned correctly within the mouth of the patient.

US-A-2009/0013993 discloses a dosage device having a storage chamber for storing medicament after dispensing from an MDI device and prior to inhalation by a patient. The dosage device includes a removable mouthpiece which includes a stem on which a lip sealing ridge having crests and troughs is formed. The lip sealing ridge provides a seal between the mouthpiece or the stem and the mouth of the patient so that the patient can inhale the medicament stored within the storage chamber through a one-way valve located between the storage chamber and mouthpiece to prevent air exhaled by the patient from re-entering the storage chamber. This reduces or prevents the risk of contamination or cross-contamination allowing different patients to use the same device by simply replacing the mouthpiece.

Whilst the dosage device described above provides means for sealing the lips of a patient to a removable mouthpiece for inhalation, the mouthpiece itself is not formed on an actuator housing for an MDI device with the result that it is more cumbersome to use. This is because the MDI device needs to be mounted to another end of the storage chamber which is remote from the mouthpiece and then the medicament is dispensed into the storage chamber prior to inhalation.

WO-A-2004/060260 discloses an MDI device which provides a mouthpiece configured for oral engagement with the patient. The outer surface of the mouthpiece contains at least one longitudinally-extending disuniformity arranged such that, when the patient engages the mouthpiece with the lips, at least one void space is created between the outer surface of the mouthpiece and the patient so as to provide an air flow channel through the at least one void space.

Pulmonary MDIs are generally operated in a “valve down” orientation. The medicament (formulated as a suspension or solution in a propellant or solvent) is in a canister that has a dispensing valve with a hollow stem that is inserted into an actuator seat. In use, the patient typically squeezes the base of the canister towards the base portion of the actuator to actuate the valve and dispense the medicament as a spray (i.e., pressure actuated dispensing). Most actuators are generally ‘L’-shaped, with a tubular housing for the container forming one branch of the ‘L’ and the mouthpiece forming the other. The base portion is often provided with features to enable it to be gripped by the patient, such as various configurations of thumb grip. The base portion is nevertheless generally planar, defining a base plane, which in use is typically horizontal.

In normal operation of an MDI device (an MDI actuator housing and a canister of medicament), a plume of medicament is produced from the exit orifice or actuator nozzle into the tubular mouthpiece portion and is inhaled by a patient through the tubular mouthpiece portion.

### SUMMARY OF THE INVENTION

In accordance with one aspect of the present invention, there is provided an actuator housing for a metered dose inhaler device, the actuator housing comprising:
a generally tubular substantially hollow first portion having a first end and a second end;
a base portion formed at the second end;
an actuator seat formed in the base portion;
an actuator nozzle formed in the actuator seat and operable for dispensing a spray of metered fluid; and

a substantially hollow tubular second portion having a longitudinal axis extending between a proximal end and a distal end and having an external surface between the proximal and distal ends, the proximal end being located adjacent the second end of the generally tubular substantially hollow first portion and the distal end defining a mouthpiece end face, said substantially hollow tubular second portion comprising a first region and a second region formed on the external surface, the second region being closer to the proximal end than the first region and being configured to define a sealing region, the first region being located adjacent the distal end and being configured to define a guidance region.

By providing a guidance region for a patient over which the teeth are to be located and a sealing region against which the lips can be sealed, a patient is intuitively encouraged to put the mouthpiece far enough into the mouth so that the lips make a comfortable seal against the sealing region whilst ensuring that a medicament spray is not obstructed by the teeth, that the mouth is properly open and that the airway is clear.

The second region may extend for at least 10 mm, more preferably at least 15 mm, in a direction substantially parallel to the longitudinal axis along the external surface from an end of the first region remote from the mouthpiece end face. The second region may comprise a continuous and generally smooth solid surface.

This spacing allows most patients to be able to seal the lips against this second region.

In one embodiment, the first region extends to within 5 mm, more preferably to within 3 mm, and most preferably to within 1 mm, of the mouthpiece end face.

This ensures that the first region is as close as possible to the mouthpiece end face so that an MDI device comprising such an actuator housing can readily be used by patients of all ages irrespective of the size of the mouth.

In one embodiment, the external surface of the second hollow tubular portion comprises a circumference and the first region extends around at least a circumferential portion thereof. In a preferred embodiment, the first region extends substantially around the circumference.

In addition, the second region extends substantially around the circumference of the second hollow tubular portion.

In one embodiment, the first region comprises at least one element protruding outwardly therefrom. The at least one element may comprise at least one circumferential rib. Two or more circumferential ribs may be preferred. Three circumferential ribs may be more preferred. The circumferential ribs may be continuous or discontinuous, and each circumferential rib may be configured to be parallel to the mouthpiece end face.

In an alternate embodiment, the at least one element comprises at least one longitudinally extending rib. A plurality of longitudinally extending ribs may be arranged around the external surface of the substantially hollow tubular second portion.

In another embodiment, the at least one element comprises a plurality of protrusions arranged in at least one row around the external surface of the substantially hollow tubular second portion. In some embodiments, the plurality of protrusions are arranged in two or more rows around the external surface of the substantially hollow tubular second portion.

By providing protrusions, correct positioning of the teeth is obtained in use so that there are no obstructions to the medicament spray when dispensed.

As an alternative to protrusions or projections, the first region comprises at least one element providing a recess in the external surface of the substantially hollow tubular second portion. The at least one element may comprise a plurality of depressions formed in the external surface. The plurality of depressions may define at least one ring around the substantially hollow tubular second portion. In some embodiments, the plurality of depressions may define two or more rings around the substantially hollow tubular second portion.

In one embodiment, the plurality of depressions comprises a plurality of flutes. Each flute preferably extends in a direction substantially parallel to the longitudinally extending axis.

The first region may comprise a plurality of apertures formed in the external surface of the substantially hollow tubular second portion. The plurality of apertures may define at least one ring around the substantially hollow tubular second portion. In some embodiments, the plurality of apertures may define two or more rings around the substantially hollow tubular second portion.

By providing depressions or apertures, it is not possible for a patient to make a seal in the first region. This ensures that the mouthpiece is inserted a sufficient distance into the mouth so that the teeth do not form an obstruction and the airway is clear.

An annular projection may be located at an end of the second region adjacent the proximal end of the substantially hollow tubular second portion, the annular projection defining an end of the second region. The annular projection is preferably aligned to be substantially parallel with the mouthpiece end face.

Such an annular projection further defines the region against which the lips should seal and prevents the mouthpiece being inserted too far into the mouth.

In accordance with another aspect of the present invention, there is provided a metered dose inhaler device comprising a canister having a metering valve, and an actuator housing as described above, the metering valve being operable for engaging with the actuator seat formed in the base portion of the actuator housing. Typically, such dispensing devices are used valve down and the valve is gravity-fed, so the valve does not have a dip tube. In dispensing devices that are used valve up, a dip tube is required. A dip tube can present a problem when dispensing formulations because of loss of prime in propellant-based formulations and/or due to inhomogeneity of the drug suspension which could take several actuations to advance up the tube.

Naturally, such a metered dose inhaler device has the advantages conferred by the actuator in that the plume of medicament dispensed from the canister via the metering valve is more likely to pass into a properly opened oral cavity, and is less likely to be obstructed by the upper front teeth, and therefore substantially reduces the loss of medicament received by the patient due to buccal and tongue deposition as well as deposition on the teeth and lips. Additionally, the metered dose inhaler device typically has no by-pass air flow channel, so all the inhalation effort of the patient is applied to direct the airflow containing the medicament spray past anatomical features in the mouth of the patient and towards the lungs.

In accordance with a further aspect of the present invention, there is provided a method of manufacturing a metered dose inhaler device comprising an actuator with a mouthpiece for delivering a spray, the actuator configured to predispose a user of the metered dose inhaler device to put the mouthpiece far enough into their mouth so that their lips make a comfortable seal against a sealing region of the mouthpiece whilst ensuring that the spray is not obstructed by their teeth.

In accordance with an additional further aspect of the present invention, there is provided a method of treatment of a pulmonary condition in a human patient, the method comprising providing a metered dose inhaler device comprising an actuator with a mouthpiece, the actuator configured to predispose a user of the metered dose inhaler device to put the mouthpiece far enough into their mouth so that their lips make a comfortable seal against a sealing region of the mouthpiece whilst ensuring that the spray is not obstructed by their teeth, the patient inserting the mouthpiece into their mouth and actuating the metered dose inhaler device while inhaling.

### BRIEF DESCRIPTION OF THE DRAWINGS

For a better understanding of the present invention, reference will now be made, by way of example, to the accompanying drawings in which:

FIG. 1a illustrates a sectioned side view of a first type of conventional actuator;

FIG. 1b illustrates a front view of the actuator of FIG. 1a;

FIG. 2a illustrates a sectioned side view of a second type of conventional actuator;

FIG. 2b illustrates a front view of the actuator of FIG. 2a;

FIG. 3 illustrates a side view of a first embodiment of an actuator in accordance with the present invention;

FIG. 4 illustrates a perspective view of the actuator of FIG. 3;

FIG. 5 illustrates a sectioned side view of the actuator of FIGS. 3 and 4;

FIG. 6 illustrates a perspective view of a second embodiment of an actuator in accordance with the present invention;

FIG. 7 illustrates a side view of a third embodiment of an actuator in accordance with the present invention;

FIG. 8 illustrates a side view of a fourth embodiment of an actuator in accordance with the present invention;

FIG. 9 illustrates a side view of a fifth embodiment of an actuator in accordance with the present invention; and

FIG. 10 illustrates a side view of sixth embodiment of an actuator in accordance with the present invention.

### DESCRIPTION OF THE INVENTION

The present invention will be described with respect to particular embodiments and with reference to certain drawings but the invention is not limited thereto. The drawings described are only schematic and are non-limiting. In the drawings, the size of some of the elements may for illustrative purposes be exaggerated and not drawn to scale.

It will be understood that the terms “vertical”, “horizontal”, “top”, “bottom”, “above”, “below”, “left”, “right” etc. as used herein refer to particular orientations of the Figures and these terms are not limitations to the specific embodiments described herein.

MDI actuators generally comprise a canister-retaining or tubular housing portion and a tubular mouthpiece portion, the tubular mouthpiece portion being angled with respect to an axis extending through the tubular housing portion. At a closed bottom end of the tubular housing portion sits a nozzle block that comprises a stem socket and an exit orifice or actuator nozzle. At the bottom of the actuator, a thumb grip is provided. The tubular mouthpiece portion may have a circular, elliptical or oblong cross-section. It has an opening that is generally greater than 13 mm in its shortest internal and external dimensions, or generally greater than 20 mm in its longest internal and external dimensions, or generally greater than 300 mm² in internal and external areas.

The term “oblong” as used herein refers to a shape which deviates from a square or circular form principally by elongation in one dimension.

In normal operation of an MDI device (an MDI actuator housing and a canister of medicament), a plume of medicament is produced from the exit orifice or actuator nozzle into the tubular mouthpiece portion and is inhaled by a patient through the tubular mouthpiece portion. However, as described above, there may be incorrect orientation or usage of the device when the medicament is being dispensed.

A major factor affecting MDI delivery efficiency is the proximity of the teeth, in particular the upper front teeth, to the actuator outlet, during inhaler use. There appears to be a large degree of variation as to the way that the manufacturers of MDI devices recommend that they be used. For example, some manufacturers instruct the patient to place the teeth around the mouthpiece, with others instructing the patient to place the lips around the mouthpiece but giving no instruction as to the position of the teeth. Unfortunately, manufacturers' instructions are renowned for not always being carefully studied or followed, and thus devices should preferably always be designed to intuitively encourage correct use.

The present invention provides for a number of actuator embodiments in which the mouthpiece is modified, when compared to the prior art actuator housings, to provide an intuitive encouragement for correct patient usage.

In the embodiments described below, the actuators encourage the patient to insert the actuator far enough into the mouth so that a seal can be provided between the lips of the patient and the mouthpiece of the actuator housing.

Ideally, in some embodiments, the actuator may also provide for a location position for at least the teeth on an external surface thereof which is both comfortable and intuitive for a patient using the MDI device of which the actuator forms a part.

Furthermore, in some embodiments, the actuator housing may provide features on or in the external surface of the mouthpiece which provide a topography or texture on which the patient's lips can only uncomfortably rest and/or which make it difficult to form a seal between the lips and that region of the mouthpiece. By providing a smooth region of external surface further along the mouthpiece, away from its open end, onto which the lips can intuitively and obviously seal, the patient may be encouraged to push the mouthpiece further into their oral cavity. Reaching the smooth sealing region with their lips in this way tends to force them to place their teeth around the outside of the mouthpiece instead of leaving them in the path of the medicament spray which will emerge from its open end.

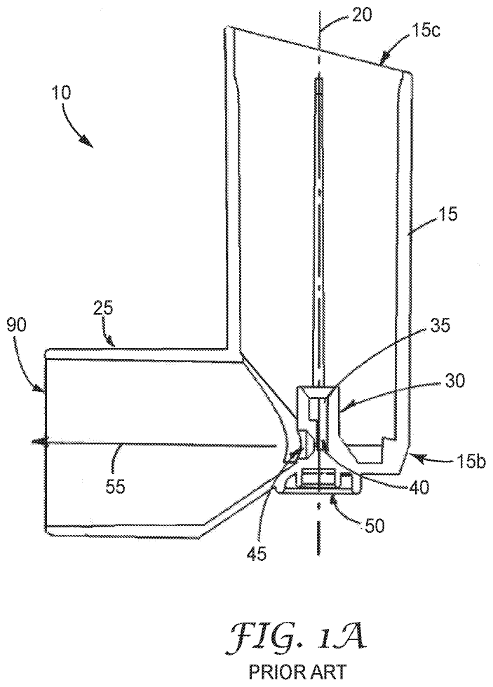

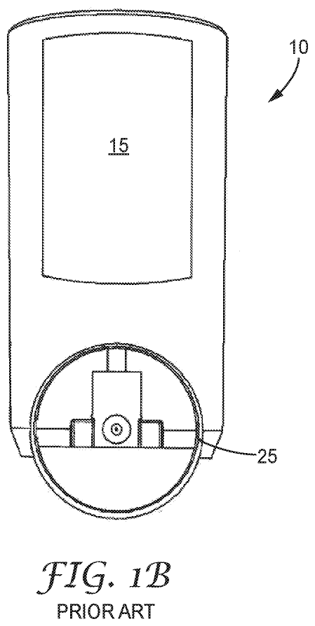

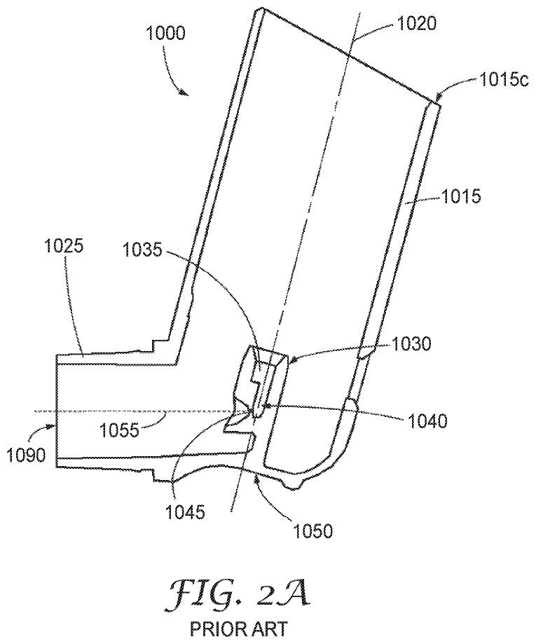

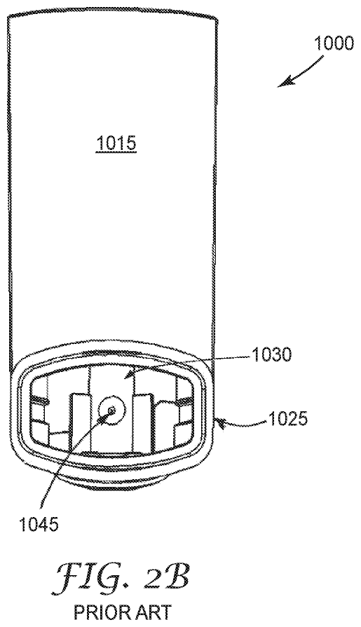

Before describing the embodiments in accordance with the present invention, two embodiments of a conventional MDI actuator housing will be described with reference to FIGS. 1a-1b and 2a-2b.

FIG. 1a illustrates a side sectioned view through a conventional MDI actuator housing 10 which comprises a substantially hollow tubular housing portion 15 having an axis 20 which extends therethrough and a substantially hollow tubular mouthpiece portion 25. The tubular housing portion 15 has an open or first end 15c and a closed or second end 15b, the closed or second end being located at a bottom or lower end of the tubular housing portion 15 and forming a base portion as shown in the Figure. Within the closed or second end 15b sits a nozzle block 30 which comprises an actuator seat or stem socket 35 in fluid communication with a sump region 40 and an exit orifice or actuator nozzle 45. On an external surface of the closed or second end 15b of the tubular housing portion 15, a thumb grip 50 is provided in the base portion which is substantially perpendicular to the axis 20.

The tubular mouthpiece portion 25 comprises a proximal end which adjoins the closed or second end 15b of the tubular housing portion 15. The tubular mouthpiece portion 25 also has an open or distal end which comprises a mouthpiece end face 90, and the tubular mouthpiece portion extends from its proximal end in a direction towards its distal end and is substantially aligned with an axis 55 which extends from a centre of the exit orifice or actuator nozzle 45 to a centre of the mouthpiece end face 90. In this embodiment, the spray is directed from the exit orifice or actuator nozzle 45 in the direction of the axis 55 as indicated by the arrow. The mouthpiece end face 90 defines a mouthpiece face plane, which, in FIG. 1a, is substantially parallel with the axis of the housing portion 15 and perpendicular to a plane in which the axis of the mouthpiece portion 25 lies. In this embodiment, the mouthpiece face plane 90 is also perpendicular to a plane in which the base portion lies.

FIG. 1b shows a front view of the actuator housing of FIG. 1a, and, features described above with reference to FIG. 1a, that are visible in this Figure, are indicated using the same reference numerals.

It will readily be understood, from the description of FIGS. 2a and 2b below, that a canister containing a medicament can be inserted into the actuator housing of FIGS. 1a and 1b in a similar way with the spray being directed substantially along the axis 55.

FIG. 2a illustrates a side sectioned view through a conventional MDI actuator housing 1000 which comprises a substantially hollow tubular housing portion 1015 having an axis 1020 which extends therethrough and a substantially hollow tubular mouthpiece portion 1025. The tubular housing portion 1015 has a nozzle block 1030 which comprises an actuator seat or stem socket 1035 in fluid communication with a sump region 1040 and an exit orifice or actuator nozzle 1045. The tubular mouthpiece portion 1025 also has an open or distal end which comprises a mouthpiece end face 1090, and the tubular mouthpiece portion extends from its proximal end in a direction towards its distal end and is substantially aligned with an axis 1055 which extends from a centre of the exit orifice or actuator nozzle 1045 to a centre of the mouthpiece end face 1090. In this embodiment, although not shown, the spray is directed from the exit orifice or actuator nozzle 1045 in the direction of the axis 1055.

FIG. 2b shows a front view of the actuator shown in FIG. 2a, and, features described above with reference to FIG. 2a, that are visible in this Figure, are indicated using the same reference numerals.

A canister containing a medicament (not shown) is mounted within the tubular housing portion 1015 so that a valve stem thereof is located within the nozzle block 1030, and in particular, within the actuator seat or stem socket 1035. An upper end of the canister extends beyond the open or first end 1015c of the tubular housing portion 1015. Downward pressure on the upper end of the canister activates a valve associated with the valve stem to release a predetermined amount of medicament into the sump region 1040 and through to the exit orifice or actuator nozzle 1045 to generate a spray (not shown) which is directed through the tubular mouthpiece portion 1025 in the direction of the axis 1055. As described above, the base portion and the thumb grip 1050 lie in a plane which provides a horizontal reference for the angle of the generated spray which is, for example, substantially 0 degrees as indicated by dotted line 1055.

In accordance with one embodiment of the present invention, an MDI actuator housing has been designed such that, when inserted into the mouth, the upper teeth rest comfortably and intuitively on an external surface of the mouthpiece portion of the actuator.

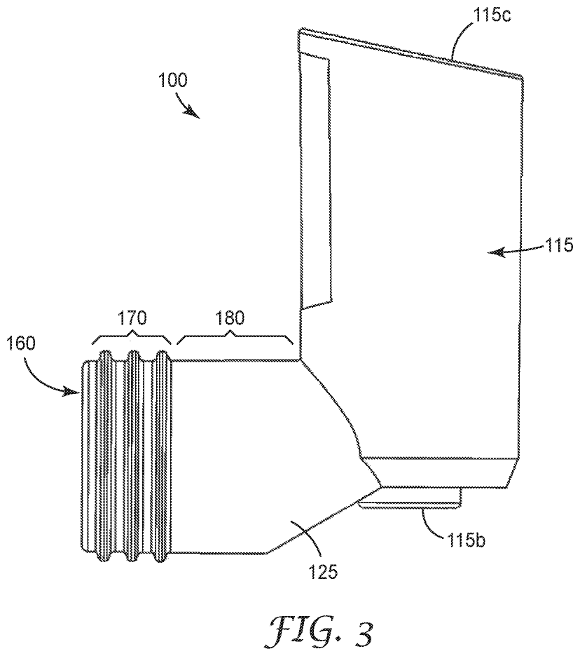

In FIG. 3, a side view through an MDI actuator housing 100 is shown which comprises a single plastic moulding in, for example, polypropylene. The housing 100 comprises a substantially hollow tubular housing portion 115 and a substantially hollow mouthpiece portion 125. The tubular housing portion 115 has an open or first end 115c and a closed or second end 115b, the closed or second end being located at a bottom or lower end of the tubular housing portion 115 and forming a base portion.

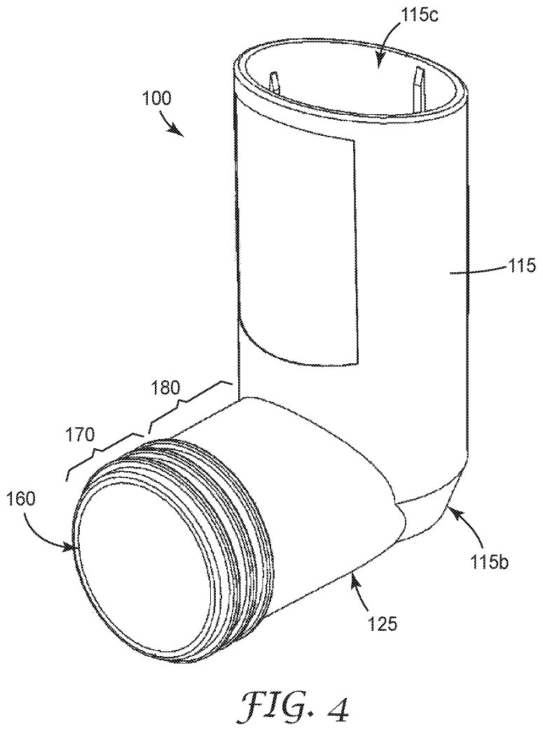

FIG. 4 illustrates a perspective view of the MDI actuator housing 100. Components which have previously been described are referenced the same and will not be described again here.

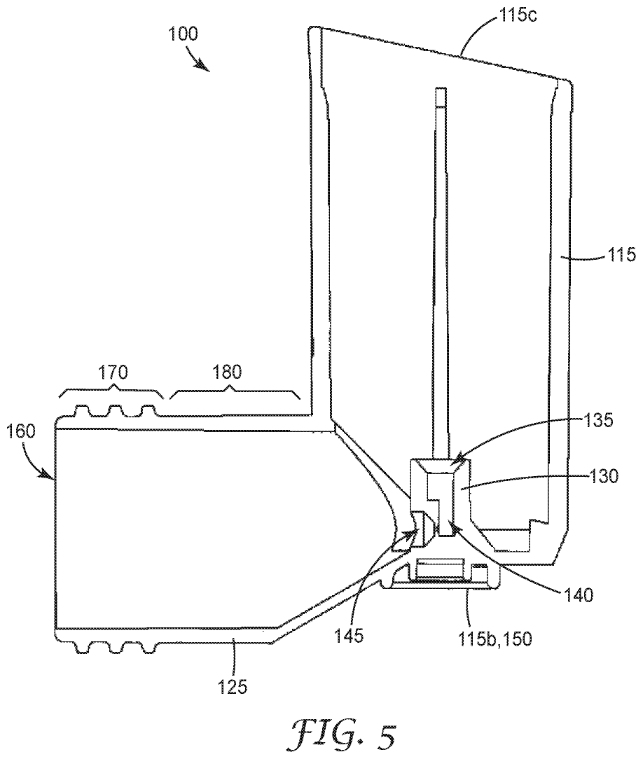

As shown in FIG. 5, a sectioned side view of the actuator housing 100 of FIGS. 3 and 4, within the closed or second end 115b sits a nozzle block 130 which comprises an actuator seat or stem socket 135 in fluid communication with a sump region 140 and an exit orifice or actuator nozzle 145. On an external surface of the closed or second end 115b forming the base portion, a thumb grip 150 is provided.

The mouthpiece portion 125 comprises a proximal end which adjoins the closed or second end 115b of the tubular housing portion 115. The tubular mouthpiece portion 125 also has an open or distal end which comprises a mouthpiece end face 160, and the mouthpiece portion extends from its distal end in a direction towards its proximal end. The mouthpiece end face 160 defines a mouthpiece face plane.

The mouthpiece portion 125 has an external surface which extends between the mouthpiece end face 160 or distal end and the proximal end adjoining the closed or second end 115b of the tubular housing portion 115.

In accordance with the present invention, the external surface comprises two regions, a guidance region and a sealing region, as will be described in more detail with respect to specific embodiments of the present invention. In particular, the sealing region is located adjacent the proximal end adjoining the closed or second end 115b of the tubular housing portion 115, and, the guidance region is located adjacent the mouthpiece end face 160 at the distal end of the mouthpiece portion 125.

The term “guidance region” as used herein refers to a region on the external surface of the mouthpiece portion 125 over which the lips of a patient needs to be located so that the lips can seal with the sealing region during dispensing of a medicament from the canister mounted within the tubular housing portion 115. In general, the surface of the guidance region is structured such that it is difficult and/or uncomfortable to form a seal when contacted with the lips of a patient. Generically, the guidance region can be referred to as a first region.

The term “sealing region” as used herein refers to a region on the external surface of the mouthpiece portion 125 against which the lips of a patient seals during dispensing of a medicament from a canister mounted in the tubular housing portion 115. Generically, the sealing region can be referred to as a second region.

In this embodiment of an actuator 100 according to the present invention, the guidance region comprises a set of three continuous circumferential ribs or rings 170 provided on the external surface of the mouthpiece 125 adjacent the mouthpiece end face 160 as shown. This set of three continuous circumferential ribs or rings 170 operates to guide a patient so that the lips are placed over this first or guidance region and into contact with the second or sealing region 180.

In use, a patient inserts the mouthpiece portion 125 into the mouth. Because the set of continuous circumferential external ribs or rings 170 are significantly sized, a typical patient is unable to make a comfortable sealing contact on them with the lips. This arrangement therefore encourages the patient to insert the mouthpiece portion 125 further into the mouth so that the lips can reach the sealing region 180 beyond the ribs or rings of the first region 170. The lips of a typical patient are not far in front of their front teeth. This means that, in order for the lips to reach the second or sealing region, the patient is forced to open the mouth sufficiently for their teeth to go round, that is, both above and below, the first or guidance region at the end of the mouthpiece portion 125.

This is because the actuator 100 effectively has the second or sealing region further along the mouthpiece portion 125 than a position to which a typical patient is able to advance the lips in front of the teeth. Therefore, in order to achieve an adequate lip seal with the external surface of the mouthpiece portion 125, the patient is intuitively forced to place the teeth around the end of the mouthpiece portion 125. In fact, the set of continuous circumferential ribs or rings 170 can also serve to provide a ledge on which the teeth can rest. Having a patient put his/her front teeth around the mouthpiece portion and on or over the first region defined by the set of circumferential ribs or rings 170 will ensure that the emerging medicament spray is unobstructed by the teeth, the mouth is adequately open and the airway is clear.

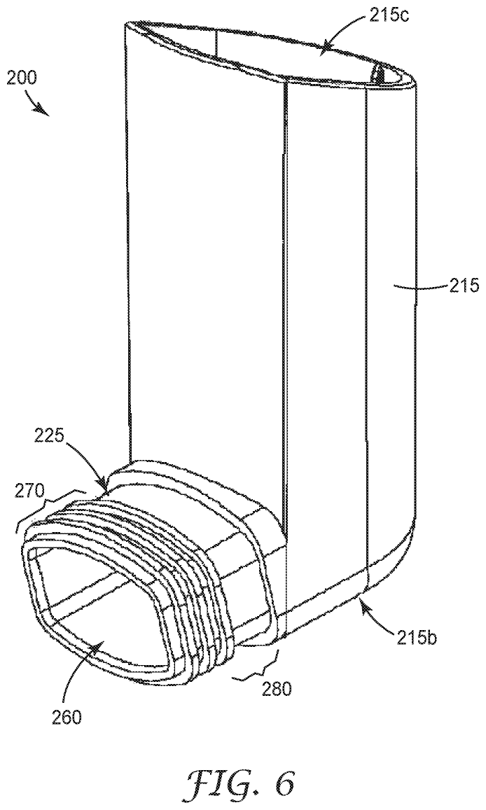

In the embodiment shown in FIGS. 3 to 5, the mouthpiece portion 125 is generally cylindrical. It should be noted that alternative geometries for the mouthpiece portion 125 are possible, for example, elliptical or rectangular cross-sections. One such alternative geometry is shown in FIG. 6.

In FIG. 6, an actuator housing 200 is shown which has a substantially hollow tubular housing portion 215 and a substantially hollow mouthpiece portion 225. The tubular housing portion 215 has an open or first end 215c and a closed or second end 215b, the closed or second end being located at a bottom or lower end of the tubular housing portion 215 and forming a base portion.

In this embodiment, both the substantially hollow tubular housing portion 215 and the substantially hollow mouthpiece portion 225 comprise substantially rectangular cross-sections. It will be appreciated however that the tubular housing portion 215 may also be circular.

As described above with reference to FIGS. 3 to 5, the mouthpiece portion 225 comprises a proximal end which adjoins the closed or second end 215b of the tubular housing portion 215. The tubular mouthpiece portion 225 also has an open or distal end which comprises a mouthpiece end face 260, and the mouthpiece portion extends from its distal end in a direction towards its proximal end. The mouthpiece end face 260 defines a mouthpiece face plane.

The mouthpiece portion 225 has an external surface which extends between the mouthpiece end face 260 or distal end and the proximal end adjoining the closed or second end 215b of the tubular housing portion 215.

In this embodiment of an actuator 200 according to the present invention, the guidance region comprises a set of three continuous circumferential ribs or rings 270 provided on the external surface of the mouthpiece 225 adjacent the mouthpiece end face 260 as shown. This set of three circumferential ribs or rings 270 operates to guide a patient so that the lips are placed over this first or guidance region and into contact with the second or sealing region 280 as described above with reference to FIGS. 3 to 5.

Preferably, the ribs or rings 270 are sufficiently sized and/or sufficiently pronounced in shape so as to prevent the formation of a comfortable seal with the lips of the patient, whilst at the same time being shaped to avoid traps for dirt, for example, not having tight inaccessible recesses or corners.

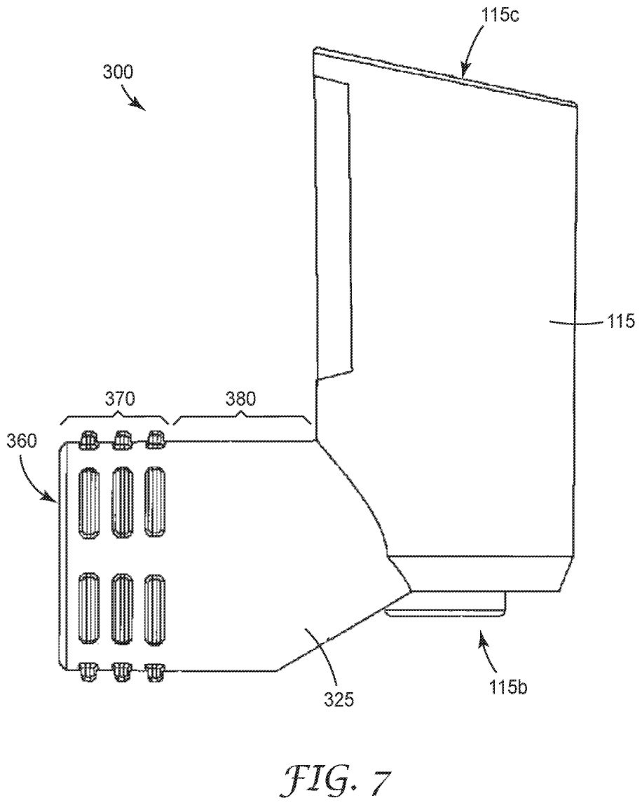

FIG. 7 illustrates a third embodiment of an actuator housing 300 in accordance with the present invention. The actuator housing 300 is similar to the actuator housing 100 as described above with reference to FIGS. 3 to 5, but with the set of continuous circumferential ribs or rings 170 replaced with a set of three discontinuous circumferential ribs or rings 370. Elements that have previously been described with reference to FIGS. 3 to 5 have the same reference numerals and will not be described again here.

As described above, the set of three discontinuous circumferential ribs of rings 370 is provided on the outside of mouthpiece portion 325, close to mouthpiece end face 360 and in a first or guidance region. Beyond the first or guidance region is a second or sealing region 380 of the external surface of the mouthpiece portion 325.

By making the circumferential ribs or rings 370 discontinuous, that is, having shorter sections, the actuator housing 300 can be more readily moulded.

In use, a typical patient is again unable to make a comfortable sealing contact with the second or sealing region of the external surface of the mouthpiece portion 325 with their lips unless the mouthpiece portion 325 is inserted far enough into the mouth, again forcing the teeth to go outside the mouthpiece portion 325 rather than to form an obstruction across the mouthpiece end face 360. Thus, again the patient is intuitively encouraged to use the actuator in such a way that the front teeth at least do not inadvertently obstruct the emerging medicament spray.

Although the discontinuous elements are shown in FIG. 7 as being regularly spaced on the external surface of the tubular mouthpiece portion 325, it will readily be appreciated that these elements may also be irregularly spaced both around the tubular mouthpiece portion and extending away from the mouthpiece end face 360.

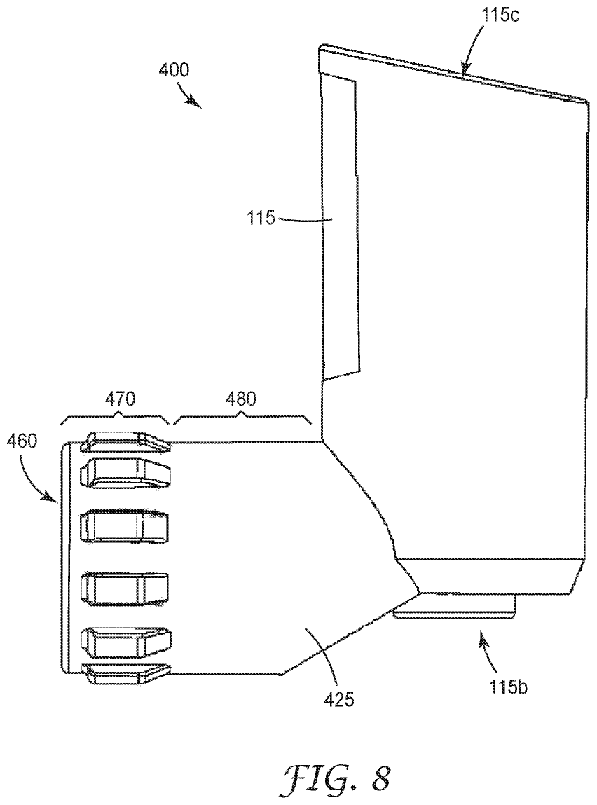

FIG. 8 is a side view of a fourth embodiment of an actuator 400 in accordance with the present invention. As before, components or features previously described above bear the same reference numbers and will not be described again here in detail.

In this embodiment, mouthpiece portion 425 includes a mouthpiece end face 460 adjacent to which a series of longitudinally extending ribs 470 is provided on the external surface of the mouthpiece portion 425, the series of longitudinally extending ribs 470 forming a first or guidance region with a second or sealing region 480 being located on the external surface between the guidance region and the proximal end of the mouthpiece portion 425.

As described above, beyond the longitudinally extending ribs 470, the external surface comprises a second or sealing region on which the patient can readily achieve a comfortable lip seal, provided that the mouthpiece has been inserted far enough into the mouth. Because of the proximity of the front teeth to the lips, the patient can only achieve this comfortable seal if their front teeth are around the external surface of the mouthpiece portion 425 of the actuator 400. The longitudinally extending ribs 470 may be shaped to form a comfortable rest for the teeth.

Although, in the illustrated embodiment, the longitudinally extending ribs 470 are regularly spaced around the tubular mouthpiece portion 425, it will readily be appreciated that the ribs may be spaced in some other way. For example, more ribs may be provided on an upper portion of the external surface of the tubular mouthpiece portion for providing a location for the upper teeth with fewer ribs being provided on a lower portion of the external surface of the tubular mouthpiece portion, and/or, the spacing between the ribs may be variable around the tubular mouthpiece portion 425 with the spaces between the ribs on the upper portion of the external surface being smaller than the spaces between the ribs on the lower portion of the external surface. In addition, the ribs do not need to be all of the same size and/or length.

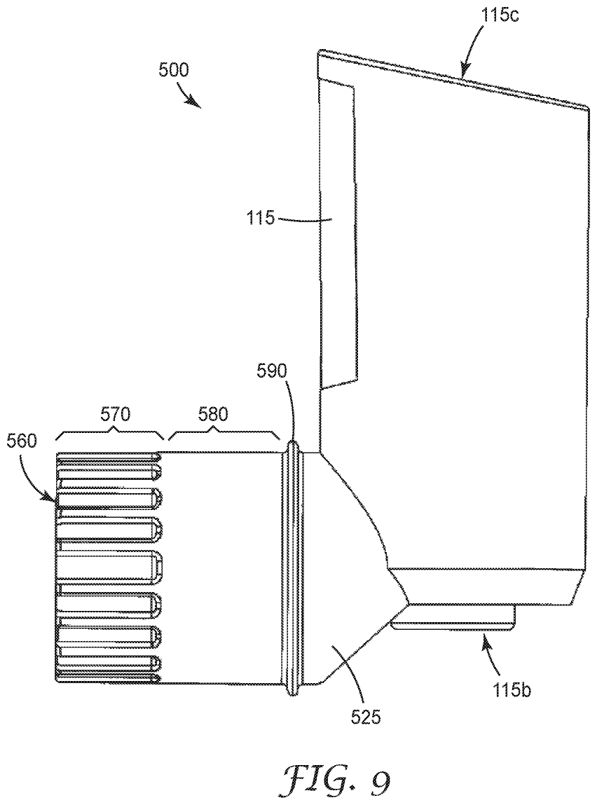

FIG. 9 is a side view of a fifth embodiment of an actuator 500 in accordance with the present invention. In this embodiment, actuator housing 500 comprises a mouthpiece portion 525 having a mouthpiece end face 560. A series of longitudinal depressions or flutes 570 is provided on the external surface of the mouthpiece portion 525 adjacent the mouthpiece end face 560 to form the first or guidance region on the external surface of the mouthpiece portion 525. Beyond these depressions or flutes 570, a second or sealing region 580 is provided in the external surface of the mouthpiece portion 525. Beyond that, an optional annular ring 590 may be provided in the form of a circumferential raised rib having a profiled cross-section to prevent the patient inserting the mouthpiece too far into the mouth. The depressions or flutes 570 are arranged to ensure that the patient cannot readily form a lip seal unless they put the mouthpiece tube far enough into the mouth so that the lips can seal against the second or sealing region of the external surface of the mouthpiece portion 525.

Although the depressions or flutes are shown in FIG. 9 as being regularly spaced on the external surface of the tubular mouthpiece portion 525, it will readily be appreciated that these depressions or flutes may also be irregularly spaced. In addition, the depressions or flutes can be the same size or different sizes.

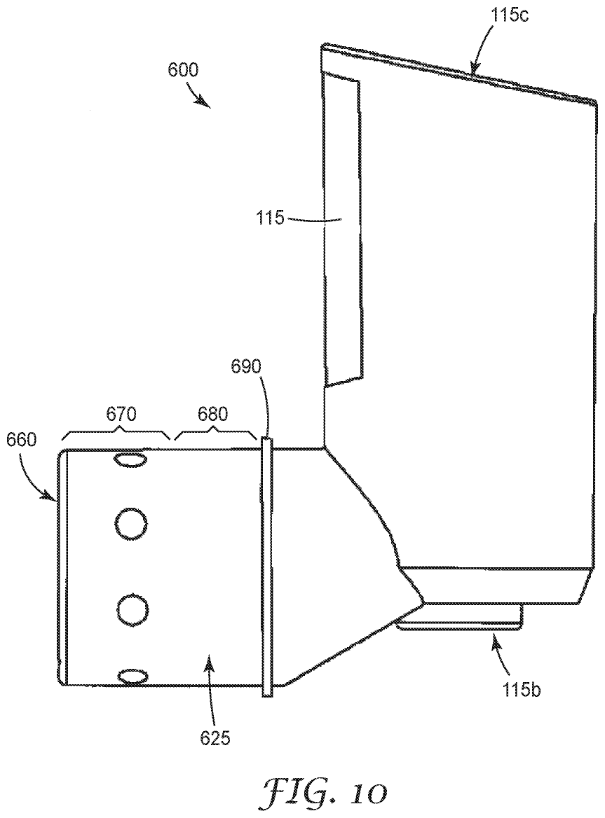

FIG. 10 is a side view of a sixth embodiment of an actuator housing 600 in accordance with the present invention. As before components or features which have been described above bear the same reference numerals and will not be described in more detail here.

In this embodiment, the actuator housing 600 comprises a mouthpiece portion 625 having a mouthpiece end face 660. A ring of apertures 670 is formed near the mouthpiece end face 660 to form the first or guidance region on the external surface of the mouthpiece portion 625. Beyond these apertures 670, a second or sealing region 680 is located. Beyond or at the end of the second or sealing region, an optional annular ring 690 in the form of a circumferential raised rib having a substantially rectangular cross-section can be provided to prevent the patient inserting the mouthpiece too far into the mouth. The apertures 670 are arranged to ensure that the patient cannot readily form a lip seal unless they put the mouthpiece tube far enough into the mouth so that the lips can seal against the second or sealing region of the external surface of the mouthpiece portion 625.

Although the apertures are shown in FIG. 10 as being regularly spaced on the external surface of the tubular mouthpiece portion 625, it will readily be appreciated that these apertures may also be irregularly spaced both around the tubular mouthpiece portion and extending away from the mouthpiece end face 660. In addition, the apertures can be the same size or different sizes.

Although the first or guidance region of this embodiment has been described as being a ring of apertures 670, it will readily be appreciated that a ring of depressions or protrusions could also be used. In addition, the annular ring 690 may comprise an annular ring 590 as described above with reference to FIG. 9.

It will readily be appreciated that the annular ring 590 of FIG. 9 and the annular ring 690 of FIG. 10 can also be used in conjunction with the embodiments shown in FIGS. 3 to 8 to limit the amount that the mouthpiece portion is inserted into the mouth.

It will also be appreciated that the annular ring 590 of FIG. 9 and the annular ring 690 of FIG. 10 may comprise different profiles to those shown in FIGS. 9 and 10.

Although the present invention has been described with reference to specific embodiments, it will readily be appreciated that other embodiments are possible.

1. An actuator housing for a metered dose inhaler device, the actuator housing comprising:
a generally tubular substantially hollow first portion having a first end and a second end;
a base portion formed at the second end;
an actuator seat formed in the base portion;
an actuator nozzle formed in the actuator seat and operable for dispensing a spray of metered fluid; and
a substantially hollow tubular second portion having a longitudinally axis extending between a proximal end and a distal end and having an external surface between the proximal and distal ends, the proximal end being located adjacent the second end of the generally tubular substantially hollow first portion and the distal end defining a mouthpiece end face, said substantially hollow tubular second portion comprising a first region and a second region formed on the external surface, the second region being closer to the proximal end than the first region and being configured to define a sealing region, the first region being located adjacent the distal end and being configured to define a guidance region.

2. An actuator housing according to claim 1, wherein the second region extends for at least 10 mm in a direction substantially parallel to the longitudinal axis along the external surface from an end of the first region remote from the mouthpiece end face.

3. (canceled)

4. An actuator housing according to claim 1, wherein the first region extends to within 5 mm of the mouthpiece end face.

5-6. (canceled)

7. An actuator housing according to claim 1, wherein the external surface of the second hollow tubular portion comprises a circumference and the first region extends around at least a circumferential portion thereof

8. An actuator housing according to claim 7, wherein the first region extends substantially around the circumference.

9. An actuator housing according to claim 8, wherein the second region extends substantially around the circumference of the second hollow tubular portion.

10. An actuator housing according to claim 1, wherein the first region comprises at least one element protruding outwardly therefrom.

11. An actuator housing according to claim 10, wherein the at least one element comprises at least one circumferential rib.

12-15. (canceled)

16. An actuator housing according to claim 10, wherein the at least one element comprises at least one longitudinally extending rib.

17. (canceled)

18. An actuator housing according to claim 10, wherein the at least one element comprises a plurality of protrusions arranged in at least one row around the external surface of the substantially hollow tubular second portion.

19. An actuator housing according to claim 1, wherein the first region comprises at least one element providing a recess in the external surface of the substantially hollow tubular second portion.

20. An actuator housing according to claim 19, wherein the at least one element comprises a plurality of depressions formed in the external surface of the substantially hollow tubular second portion.

21. An actuator housing according to claim 20, wherein the plurality of depressions defines at least one ring around the substantially hollow tubular second portion.

22-23. (canceled)

24. An actuator housing according to claim 1, wherein the first region comprises a plurality of apertures formed in the external surface of the substantially hollow tubular second portion.

25. An actuator housing according to claim 24, wherein the plurality of apertures defines at least one ring around the substantially hollow tubular second portion.

26. An actuator housing according to claim 1, further comprising an annular projection located at an end of the second region adjacent the proximal end of the substantially hollow tubular second portion, the annular projection defining an end of the second region.

27. An actuator housing according to claim 26, wherein the annular projection is aligned to be substantially parallel with the mouthpiece end face.

28. A metered dose inhaler device comprising a canister having a metering valve, and an actuator housing according to claim 1, the metering valve being operable for engaging with the actuator seat formed in the base portion of the actuator housing.

29. A method of manufacturing a metered dose inhaler device comprising an actuator with a mouthpiece for delivering a spray, the actuator configured to predispose a user of the metered dose inhaler device to put the mouthpiece far enough into their mouth so that their lips make a comfortable seal against a sealing region of the mouthpiece whilst ensuring that the spray is not obstructed by their teeth.

30. A method of treatment of a pulmonary condition in a human patient, the method comprising providing a metered dose inhaler device comprising an actuator with a mouthpiece, the actuator configured to predispose a user of the metered dose inhaler device to put the mouthpiece far enough into their mouth so that their lips make a comfortable seal against a sealing region of the mouthpiece whilst ensuring that the spray is not obstructed by their teeth, the patient inserting the mouthpiece into their mouth and actuating the metered dose inhaler device while inhaling.
